(19) United States
(12) Patent Application Publication
(10) Pub. No.: US 2011/0224656 A1
(43) Pub. Date: Sep. 15, 2011

(54) METHOD AND APPARATUS FOR SELECTIVE PHOTOTHERMOLYSIS OF VEINS

(75) Inventors: Richard Rox Anderson, Boston, MA (US); Iris Rubin, Potomac, MD (US); William A. Farinelli, Danvers, MA (US)

(73) Assignee: THE GENERAL HOSPITAL CORPORATION, BOSTON, MA (US)

(21) Appl. No.: 12/936,348

(22) PCT Filed: Apr. 1, 2009
(86) PCT No.: PCT/US09/39153

§ 371 Date: Jun. 1, 2011

Related U.S. Application Data
(60) Provisional application No. 61/042,249, filed on Apr. 3, 2008.

(51) Int. Cl. A61B 18/20

(52) U.S. Cl. 606/3

(57) ABSTRACT

Exemplary embodiments of the present disclosure provide method and apparatus for providing electromagnetic radiation to a biological tissue that may be selectively absorbed by venous structures as compared to arteries. For example, a wavelength of the electromagnetic radiation can be selected based on absorptivity of the radiation by oxygenated hemoglobin, deoxygenated hemoglobin, and/or met-hemoglobin. The wavelength can be, e.g., between about 685 nm and about 705 nm, or between about 690 nm and about 700 nm, or about 694 nm. The exemplary methods and apparatus can be used, e.g., for photothermolysis treatment of venous structures such as port wine stains or varicose veins, while reducing or avoiding undesirable damage to nearby arteries in the irradiated tissue.

### CROSS REFERENCE TO RELATED APPLICATION

The present application claims priority from U.S. Provisional Patent Application Ser. No. 61/042,249 filed Apr. 3, 2008, the disclosure of which is incorporated herein by reference in its entirety.

### FIELD OF THE DISCLOSURE

The present disclosure relates to exemplary embodiments of methods and apparatus for a selective photocoagulation of veins, for example, a treatment of port wine stains or varicose veins, while avoiding significant thermal damage to arteries.

### BACKGROUND INFORMATION

A blood vessel can be any vascular structure, e.g., an artery, a vein, or a capillary. Perfusion (blood flow) can help to maintain a blood vessel in a healthy condition. Perfusion of blood is an important function of blood vessels. Conversely, when a vessel is closed off and perfusion stops, the vessel may eventually thrombose, die, and/or degrade. It may be desirable in certain situations to reduce or eliminate perfusion in certain blood vessels, e.g., in venous malformations, for therapeutic and/or cosmetic purposes. Non-invasive methods for reducing perfusion using, e.g., photothermolysis can be provided to make selective use of this natural process.

Irregularities in blood vessel structures can be detrimental to health, and may also be aesthetically undesirable. A dilated or malformed vein may be associated with one or more of a variety of disease conditions such as, e.g., port wine stains or varicose veins. For example, port wine stains may include post-capillary venules. Formation of port wine stains can begin in infancy, and they may both thicken and darken in color with time. In addition to being disfiguring, port wine stains can also have adverse psychosocial effects.

A conventional treatment for port wine stains may use, e.g., a pulsed dye laser at a wavelength of 595 nm. However, a success rate for complete clearance of port wine stains can be low when using conventional treatment modalities such as the 595 nm pulsed dye laser, which can result in part from an inadequate depth of penetration by the laser energy. Deep vessels can also be targeted, e.g., using a 1064 nm Nd:YAG laser treatment for port wine stains. However, a wavelength of 1064 nm can be more strongly absorbed by arterial blood (which contains primarily oxygenated hemoglobin, “HbO2”), than by venous blood (which contains a mixture of HbO2 and deoxygenated hemoglobin, “Hb”).

Accordingly, use of the Nd:YAG laser to treat port wine stains can create undesirable arterial damage, causing tissue necrosis and scarring, and may be dangerous to a patient. Although radiation from a 595 nm pulsed dye laser may be absorbed slightly more by deoxygenated hemoglobin (Hb) than by oxygenated hemoglobin (HbO2), a fluence of the pulsed dye laser may be limited by potential thermal damage to arteries.

Varicose veins can refer to dilated, often tortuous veins that may result from defective structure or function of the valves of the veins, from intrinsic weakness of a vein wall, or from arteriovenous fistulas. Varicose veins can be categorized as superficial or deep. Superficial varicose veins may be primary, e.g., originating in the superficial system, or secondary, e.g., resulting from deep venous insufficiency and incompetent perforating veins, or from deep venous occlusions that can cause enlargement of superficial veins serving as collateral veins.

Superficial varicose veins can have an undesirable cosmetic appearance. Conventional treatments for superficial varicose veins may include, for example, sclerotherapy or surgical therapy. Sclerotherapy can include an injection of a sclerosing solution such as hypertonic saline or surfactants into blood vessels of interest, which can result in deformation of the vascular structure. Surgical therapy may involve extensive ligation and/or stripping of greater and lesser saphenous veins. However, administration of such therapies can require a high degree of technical skill. Also, a fear of needles and/or surgical procedures may prevent many patients from seeking such treatments.

Lasers and other light sources can be used in photothermolysis therapy to treat dilated blood vessels, such as superficial varicose veins. Photothermolysis treatment techniques are described, e.g., in U.S. Pat. No. 5,558,667. Absorbed light, which may be provided in a form of pulses, can be used to damage the vessels while sparing surrounding tissues. For example, irradiation of a blood vessel with an electromagnetic radiation leads to absorption of energy by blood components contained therein and subsequent heating of the vessel. The heated vessel may thrombose and collapse, which can produce desired therapeutic effects for treatment of venous malformations. However, nearby arteries may also be damaged by such photothermolysis techniques, which can lead to partial or complete closure of the arteries, necrosis of adjacent tissue, and unwanted scarring.

Reperfusion of treated blood vessels can reduce the effectiveness of photothermolysis treatment. Multiple treatments may be preferred to reduce a likelihood of reperfusion of a treated vessel. For example, such reperfusion may be more probable if the amount of applied energy is limited to avoid unwanted damage to nearby arteries. High costs, number of treatments, and risk of post-treatment pigmentation are other negative factors which can be associated with photothermolysis therapy.

Superficial varicose veins can be treated using sclerotherapy, which is often effective but painful. Sclerotherapy can produce undesirable side effects including, e.g., hyperpigmentation, matting, and/or ulceration. Various lasers can be used for treating ectatic leg veins, such as a pulsed dye laser operating at a wavelength of 595 nm, an alexandrite laser at 755 nm, a diode laser at 800/810 nm, or a NdYag laser at 1064 nm. However, treatment of veins using such conventional lasers may not be very effective and/or may produce undesirable side effects. Phototreatment of veins using lasers or other sources of electromagnetic radiation, such as intense pulsed light (“IPL”) sources, may also generate unwanted thermal damage in nearby arteries.

Thermal damage of veins such as, e.g., varicose veins, or post-capillary venules which may be present in port wine stains, using certain photothermolysis techniques can provide desirable effects. However, conventional photothermolysis techniques may also produce unwanted thermal damage to nearby arteries, which can lead to unwanted effects, such as local necrosis and scarring.

Accordingly, there may be a need for a laser or other source of electromagnetic energy that is more selective for veins than arteries for treatment of various blood vessel conditions.

### SUMMARY OF EXEMPLARY EMBODIMENTS

Exemplary embodiments of the present disclosure are directed to methods and apparati that can provide selective photothermolysis of venous lesions using, e.g., a laser, IPL source, or other source of electromagnetic radiation which can facilitate a relative sparing of arteries.

In one exemplary embodiment, a method can be provided for applying a particular electromagnetic radiation to a biological tissue, such as skin. Characteristics of the radiation can be selected such that the radiation can be selectively absorbed, e.g., by one or more veins or venous structures present in the tissue as compared with arteries that may be present therein. For example, the optical radiation can include one or more wavelengths between about 685 nm and about 705 nm, or between about 690 nm and about 700 nm, or a wavelength of about 694 nm. The radiation can be provided by, e.g., a pulsed dye laser, a ruby laser, or another type of laser. A filtered intense pulsed light source can also be used.

Veins and/or vascular lesions which can be treated in accordance with exemplary embodiments of the present disclosure can include, but are not limited, to varicose veins and port wine stains. Venous malformations in organs other than the skin can also be treated using exemplary embodiments of the present disclosure.

In further exemplary embodiments of the present disclosure, an apparatus can be provided that is configured to apply the particular radiation to a target region of tissue that may contain, e.g., one or more veins or other vascular structures. Such exemplary apparatus can be used, for example, for a photothermolysis procedure. Characteristics of the particular radiation can be selected to facilitate selective absorption of applied radiation by veins as compared with arteries, which can avoid unwanted arterial damage. The exemplary apparatus can include, e.g., a radiation source, control circuitry, and an optional optical arrangement structured and/or configured to direct the radiation toward particular regions of the tissue being treated. The exemplary apparatus can also include a cooling arrangement configured to cool a surface of the tissue region being treated.

The exemplary apparatus can also be structured and/or configured to detect a presence of purpura in the target region, and to stop or reduce an intensity and/or fluence of the applied radiation if such purpura is detected. This detection procedure can facilitate an avoidance of non-specific damage within the target region that can arise by an absorption of radiation energy by blood outside of the blood vessels that can form the purpura.

A plurality of pulses can also be directed onto the target tissue, where a fluence and/or duration of each pulse may be less than a critical value needed to create purpura in the target tissue. Such exemplary pulse sequence can provide an improved overall selectivity of absorption of the radiation by blood vessels within the target tissue. In a further exemplary embodiment, a plurality of pulses can be directed onto the target tissue, where a fluence and/or duration of each pulse can be less than the fluence and/or duration of each previous pulse. Such pulse sequence can provide an improved treatment of the blood vessels within the target tissue. For example, longer initial pulses in such exemplary pulse sequence can preferentially affect larger blood vessels, whereas subsequent pulses having lower energies can also be effective in treating smaller vessels within the target tissue.

These and other objects, features and advantages of the present disclosure will become apparent upon reading the following detailed description of exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying figures showing illustrative embodiments, results and/or features of the exemplary embodiments of the present disclosure, in which:

FIG. 1 is an exemplary graph of absorption of electromagnetic energy by Hb and HbO2 as a function of wavelength of the energy, together with an absorption ratio of Hb and HbO2;

FIG. 2 is an exemplary graph of a wavelength-dependent absorption ratio of electromagnetic energy by Hb and HbO2, and a calculated absorption ratio of venous and arterial blood, as a function of wavelength of electromagnetic radiation;

FIG. 3 is an exemplary graph of experimentally-observed average threshold energies for a venous blood mixture and an arterial blood mixture;

FIG. 4 is an exemplary graph of experimentally observed ratios of artery/vein threshold energy, and predicted selectivity of venous blood compared to arterial blood and met-hemoglobin based on absorption data for various hemoglobin species; and

FIG. 5 is a schematic diagram of an exemplary apparatus that may be used in accordance with exemplary embodiments of the present disclosure.

Throughout the drawings, the same reference numerals and characters, unless otherwise stated, are used to denote like features, elements, components, or portions of the illustrated embodiments. Moreover, while the present disclosure will now be described in detail with reference to the figures, it is done so in connection with the illustrative embodiments and is not limited by the particular embodiments illustrated in the figures. It is intended that changes and modifications can be made to the described embodiments without departing from the true scope and spirit of the present disclosure as defined by the appended claims.

### DETAILED DESCRIPTION OF EXEMPLARY EMBODIMENTS

In an exemplary embodiment of the present disclosure, a photothermolysis procedure can be provided in which vascular structures may be irradiated with a particular electromagnetic radiation. Such particular radiation can be configured to preferentially generate certain interactions and/or effects with some blood vessels while reducing a presence of undesirable effects in other blood vessels.

For example, exemplary embodiments of the present disclosure can provide methods, e.g., for performing photothermolysis, that include providing an electromagnetic radiation that can be more strongly absorbed by certain types of blood vessels. For example, a particular radiation can be directed to a target region of tissue to be treated. The difference in absorption strength or absorption efficiency of the radiation can, for example, facilitate a generation of thermal damage in particular veins while avoiding a generation of a significant amount of thermal damage in certain nearby arteries. In one exemplary embodiment of the present disclosure, such radiation can be directed to a target area tissue and may cause thermal damage to certain veins located within the target volume (e.g., perfusion may be reduced or eliminated in these veins), while certain arteries within the target volume may be spared from significant thermal damage (e.g., perfusion may be maintained in these arteries).

In a further exemplary embodiments of the present disclosure, an apparatus can be provided which can effectuate a particular electromagnetic radiation, e.g., that can be used in a photothermolysis procedure, where the radiation can generate thermal damage, e.g., in venous structures while avoiding generation of a significant thermal damage in arteries, when the radiation is directed onto tissue that can contain both types of blood vessels. The exemplary apparatus can be structured to provide electromagnetic radiation having particular characteristics that may allow an increased selectivity of absorption by certain tissue structures, e.g., by veins as compared with arteries, and thereby may provide some selectivity in preferentially heating and thermally damaging veins while avoiding significant heating or damage of nearby arteries.

In exemplary embodiments of the present disclosure, characteristics of the particular radiation can be selected based on several factors. For example, blood vessels may typically contain red blood cells that are rich in hemoglobin. The hemoglobin can act as a chromophore that may absorb certain radiation, and which may be largely absent in surrounding tissues, such as the dermis or fatty tissue. Accordingly, hemoglobin can be a suitable target for selective absorption of energy within blood vessels that can facilitate targeted heating and/or damage to the blood vessels.

For example, deoxygenated hemoglobin (e.g., Hb, or “deoxyhemoglobin”) and/or oxygenated hemoglobin (HbO2, or “oxyhemoglobin”) can absorb certain electromagnetic radiation, which can generate a local heating of blood vessels containing these substances. Such heating can induce thermal damage in the blood vessels, and can also reduce or eliminate a perfusion of blood therein. The absorption efficiency of both Hb and HbO2 varies with a wavelength of the applied electromagnetic radiation. Further, the absorption coefficients of Hb and HbO2 can be different from each other, and each absorption coefficient may also vary with a different wavelength of electromagnetic radiation.

Arterial blood can often contain predominantly oxygenated hemoglobin (HbO2), whereas venous blood may typically include a mix of both oxygenated and deoxygenated hemoglobin (HBO2 and Hb). Met-hemoglobin (“metHb”) can refer to a form of hemoglobin in which the iron in the heme group is in an Fe³⁺ state rather than an Fe²⁺ state, e.g., of normal hemoglobin. Met-hemoglobin can be formed from Hb and/or HbO2 in measurable quantities, e.g., in blood that is exposed to electromagnetic radiation. Formation of metHb is described, e.g., in Randeberg et al., Lasers Surg. Med., vol. 34(5), pp. 414-9 (2004). Absorption efficiency of electromagnetic radiation by metHb may also vary with the wavelength of the radiation.

Veins can contain, for example, approximately 30% deoxyhemoglobin (Hb) and 70% oxyhemoglobin (HbO2); precise composition values can depend on factors such as, e.g., a particular organ associated with the vein and a metabolic need for oxygen extraction. Arteries, which can carry oxygenated blood to various parts of the body, may contain primarily HbO2.

A ratio of the absorption coefficient for a particular electromagnetic radiation by a vein (“Uvein”) to the absorption coefficient for the particular electromagnetic radiation by an artery (“Uartery”) can be estimated mathematically using these exemplary compositions of venous and arterial blood. This ratio can be used, e.g., as a measure of venous selectivity of energy absorption for a particular wavelength of radiation.

The absorption coefficient ratio Uvein/Uartery (e.g., absorption by veins/absorption by arteries) may be expressed in part using the absorption coefficients for Hb and HbO2 (“UHb” and “UHbO2,” respectively). The absorption of the particular radiation by a vein, Uvein, can be approximated by the following expression:

Uvein=UHbO2*Sa(v)+UHb*(1−Sa(v)), (1)

where Sa(v) can represent a fractional saturation of oxygen in venous blood, e.g., a ratio of oxygenated hemoglobin, HbO2, to a total amount of hemoglobin (e.g., Hb+HbO2) that may be present in the vein. In a similar manner, absorption of the particular radiation by an artery, Uartery, may be expressed in part by the equation:

Uartery=UHbO2*Sa(a)+UHb*(1−Sa(a)), (2)

where Sa(a) can represent the fractional saturation of oxygen in arterial blood, e.g., a ratio of oxygenated hemoglobin, HbO2, to a total amount of hemoglobin (e.g., Hb+HbO2) that may be present in the artery.

As described herein, a typical value of Sa(v) can be about 0.7 (e.g., a vein may contain approximately 70% oxygenated blood), and a value of Sa(a) can be about 1.0 (e.g., blood in an artery can be substantially fully oxygenated). Using these exemplary values, the expressions for Uvein and Uartery in Eqs. (1) and (2) can be provided as:

Uvein=0.7UHbO2+0.3UHb, (3)

and

Uartery=UHbO2. (4)

The ratio of absorption of radiation by a vein to absorption by an artery, Uvein/Uartery, can then be expressed as:

Uvein/Uartery=(0.7UHbO2+0.3UHb)/UHbO2. (5)

The absorption ratio Uvein/Uartery described above, which is based on a relative composition of deoxyhemoglobin (Hb) and oxyhemoglobin (HbO2) in veins and arteries, can be used for determining a measure of venous selectivity.

Table 1 provides data indicating numerical values of absorption coefficients of electromagnetic radiation, having wavelengths between 620 nm and 680 nm, by Hb (e.g., UHb) and by HbO2 (e.g., UhbO2). The data of Table 1 indicates a maximum absorption ratio UHb/UHbO2 of about 10.23 occurs within the provided wavelength range at a wavelength of about 654 nm. For example, this absorption ratio can be greater than 10 for wavelengths between about 644 nm and 662 nm. Further, the UHb/UHbO2 absorption ratio is greater than about 9 for wavelengths between about 634 nm and 676 nm.

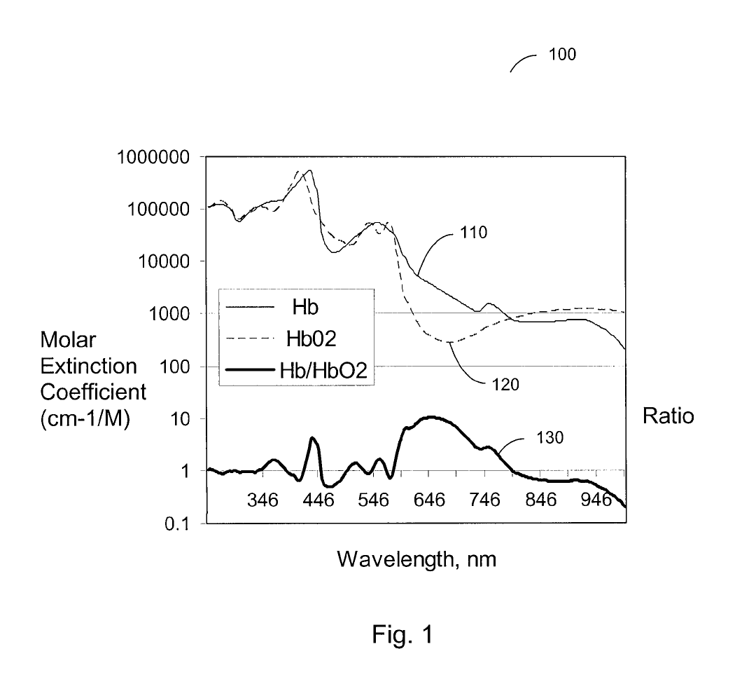

FIG. 1 is an exemplary graph 100 showing values of an absorption coefficient of deoxyhemoglobin 110 and of oxyhemoglobin 120 as a function of electromagnetic energy wavelength. In addition, FIG. 1 shows the values of a ratio 130 of the absorption of the electromagnetic energy by deoxyhemoglobin 110 to the absorption by oxyhemoglobin 120. This exemplary graph indicates that the Hb/HbO2 absorption ratio is larger for electromagnetic radiation having wavelengths between about 600 nm and 700 nm.

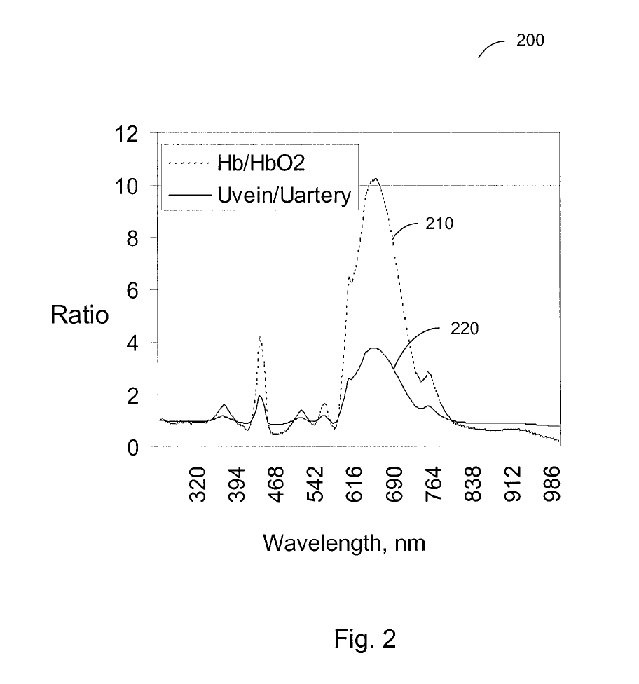

FIG. 2 is an exemplary graph 200 showing values of a ratio 210 of deoxyhemoglobin absorption, UHb, to oxyhemoglobin absorption UHbO2, over a range of wavelengths of electromagnetic radiation. FIG. 2 also shows values of a calculated selectivity ratio 220, e.g., Uvein/Uartery, as a function of radiation wavelength based on these two forms or types of hemoglobin. The absorption selectivity of veins as compared to arteries, which can be calculated using Eq. (5) as described herein, likely exhibits a local maximum value at a wavelength near 650 nm, and this ratio can decrease with an increasing or decreasing of the wavelength.

Values of Uvein and Uartery, together with a selectivity ratio Uvein/Uartery, are provided in Table 2 for radiation wavelengths between 620 nm and 680 nm. This data indicates that the selectivity ratio Uvein/Uartery, calculated based on Eq. (5), has a maximum value of about 3.77 at a wavelength of about 654 nm. This ratio remains above 3.7 for wavelengths between about 644 nm and 662 nm, and is greater than 3.6 for wavelengths between about 638 nm and 668 nm. This selectivity ratio is also greater than 3.3 for wavelengths between about 632 nm and about 680 nm.

The calculated absorption coefficient Uvein/Uartery provided in Table 2 herein and shown in the graph 200 of FIG. 2 can be based on experimentally measured absorption coefficients UHb and UHbO2 for deoxygenated hemoglobin and oxygenated hemoglobin, respectively. These theoretical values do not include, for example, any effects of absorption of electromagnetic radiation by met-hemoglobin that may be formed upon irradiation of the hemoglobin compounds, e.g., when directing electromagnetic radiation onto blood vessels.

Table 3 herein provides data that includes numerical values of millimolar absorptivities, by metHb (UmetHb), of electromagnetic radiation having wavelengths between 450 nm and 1000 nm. The data of Table 3 indicates that the absorptivity of metHb, UmetHb, decreases significantly at wavelengths greater than about 660 nm, and appears to reach a minimum value at around 700 nm before rising again with increasing wavelength.

Table 3 also includes an absorption coefficient ratio Uvein/UmetHb, which indicates the relative absorptivity of an estimated 70% HbO2/30% Hb mix in a vein (with no metHb assumed to be present) to the absorptivity of met-hemoglobin, which may be formed upon irradiation of such hemoglobin compounds. The data in Table 3 indicate that the ratio Uvein/UmetHb attains a large value at about 578 nm, decreases with increasing wavelength to about 630 nm, and then reaches a local maximum value between about 680 nm and 700 nm, before decreasing with further increasing wavelength.

It may not be easy to accurately predict or estimate a relative amount of metHb that may be formed from Hb and/or HbO2 when blood vessels containing these types or forms of hemoglobin are irradiated by electromagnetic radiation having various wavelengths, fluences, and/or intensities. However, experiments can be performed to evaluate an effect of metHb formation and presence in blood vessels on absorption selectivities under particular conditions.

For example, experiments have been performed, e.g., to measure wavelength-dependent fluence thresholds for photocoagulation of whole human blood samples having oxygen saturation levels that may be representative of arterial and venous blood. Electromagnetic radiation was controllably directed onto such blood samples, provided in glass capillary tubes, using pulsed dye lasers configured to emit electromagnetic radiation at 585 nm, 590 nm, 595 nm, 600 nm, and 633 nm, a ruby laser emitting radiation at 694 nm, an alexandrite laser configured to emit radiation at 755 nm, and a 1064 nm Nd:YAG laser. A pulse width of about 1.5 ms was used for each wavelength examined.

TABLE 1
Exemplary wavelength-dependent absorptivity and absorption
selectivity of electromagnetic radiation by deoxygenated hemoglobin,
Hb, and oxygenated hemoglobin, HbO2.

| Wavelength | UHb | UHbO2 | |
|---|---|---|---|
| (nm) | (cm-1/M) | (cm-1/M) | UHb/UHbO2 |
| 620 | 942 | 6509.6 | 6.910403397 |
| 622 | 858 | 6193.2 | 7.218181818 |
| 624 | 774 | 5906.8 | 7.631524548 |
| 626 | 707.6 | 5620 | 7.942340305 |
| 628 | 658.8 | 5366.8 | 8.146326655 |
| 630 | 610 | 5148.8 | 8.440655738 |
| 632 | 561.2 | 4930.8 | 8.786172488 |
| 634 | 512.4 | 4730.8 | 9.232630757 |
| 636 | 478.8 | 4602.4 | 9.612364244 |
| 638 | 460.4 | 4473.6 | 9.716768028 |
| 640 | 442 | 4345.2 | 9.830769231 |
| 642 | 423.6 | 4216.8 | 9.954674221 |
| 644 | 405.2 | 4088.4 | 10.08983218 |
| 646 | 390.4 | 3965.08 | 10.15645492 |
| 648 | 379.2 | 3857.6 | 10.17299578 |
| 650 | 368 | 3750.12 | 10.19054348 |
| 652 | 356.8 | 3642.64 | 10.20919283 |
| 654 | 345.6 | 3535.16 | 10.22905093 |
| 656 | 335.2 | 3427.68 | 10.22577566 |
| 658 | 325.6 | 3320.2 | 10.19717445 |
| 660 | 319.6 | 3226.56 | 10.09561952 |
| 662 | 314 | 3140.28 | 10.00089172 |
| 664 | 308.4 | 3053.96 | 9.902594034 |
| 666 | 302.8 | 2967.68 | 9.800792602 |
| 668 | 298 | 2881.4 | 9.669127517 |
| 670 | 294 | 2795.12 | 9.507210884 |
| 672 | 290 | 2708.84 | 9.340827586 |
| 674 | 285.6 | 2627.64 | 9.200420168 |
| 676 | 282 | 2554.4 | 9.058156028 |
| 678 | 279.2 | 2481.16 | 8.886676218 |
| 680 | 277.6 | 2407.92 | 8.674063401 |

TABLE 2
Exemplary calculated wavelength-dependent absorptivity of
electromagnetic radiation by a vein (Uvein) and by an artery (Uartery),
together with an absorption selectivity ratio Uvein/Uartery.

| Wavelength | Uvein | Uartery | |
|---|---|---|---|
| (nm) | (cm-1/M) | (cm-1/M) | Uvein/Uartery |
| 620 | 2612 | 942 | 2.77 |
| 622 | 2459 | 858 | 2.87 |
| 624 | 2314 | 774 | 2.99 |
| 626 | 2181 | 708 | 3.08 |
| 628 | 2071 | 659 | 3.14 |
| 630 | 1972 | 610 | 3.23 |
| 632 | 1872 | 561 | 3.34 |
| 634 | 1778 | 512 | 3.47 |
| 636 | 1716 | 479 | 3.58 |
| 638 | 1664 | 460 | 3.62 |
| 640 | 1613 | 442 | 3.65 |
| 642 | 1562 | 424 | 3.69 |
| 644 | 1510 | 405 | 3.73 |
| 646 | 1463 | 390 | 3.75 |
| 648 | 1423 | 379 | 3.75 |
| 650 | 1383 | 368 | 3.76 |
| 652 | 1343 | 357 | 3.76 |
| 654 | 1302 | 346 | 3.77 |
| 656 | 1263 | 335 | 3.77 |
| 658 | 1224 | 326 | 3.76 |
| 660 | 1192 | 320 | 3.73 |
| 662 | 1162 | 314 | 3.70 |
| 664 | 1132 | 308 | 3.67 |
| 666 | 1102 | 303 | 3.64 |
| 668 | 1073 | 298 | 3.60 |
| 670 | 1044 | 294 | 3.55 |
| 672 | 1016 | 290 | 3.50 |
| 674 | 988 | 286 | 3.46 |
| 676 | 964 | 282 | 3.42 |
| 678 | 940 | 279 | 3.37 |
| 680 | 917 | 278 | 3.30 |

TABLE 3
Exemplary calculated wavelength-dependent
absorptivity of electromagnetic radiation by a vein (Uvein) and by
an artery (Uartery), together with an absorption selectivity ratio
Uvein/Uartery.

| Wavelength | Uvein | Uartery | |
|---|---|---|---|
| (nm) | (cm-1/M) | (cm-1/M) | Uvein/Uartery |
| 620 | 2612 | 942 | 2.77 |
| 622 | 2459 | 858 | 2.87 |
| 624 | 2314 | 774 | 2.99 |
| 626 | 2181 | 708 | 3.08 |
| 628 | 2071 | 659 | 3.14 |
| 630 | 1972 | 610 | 3.23 |
| 632 | 1872 | 561 | 3.34 |
| 634 | 1778 | 512 | 3.47 |
| 636 | 1716 | 479 | 3.58 |
| 638 | 1664 | 460 | 3.62 |
| 640 | 1613 | 442 | 3.65 |
| 642 | 1562 | 424 | 3.69 |
| 644 | 1510 | 405 | 3.73 |
| 646 | 1463 | 390 | 3.75 |
| 648 | 1423 | 379 | 3.75 |
| 650 | 1383 | 368 | 3.76 |
| 652 | 1343 | 357 | 3.76 |
| 654 | 1302 | 346 | 3.77 |
| 656 | 1263 | 335 | 3.77 |
| 658 | 1224 | 326 | 3.76 |
| 660 | 1192 | 320 | 3.73 |
| 662 | 1162 | 314 | 3.70 |
| 664 | 1132 | 308 | 3.67 |
| 666 | 1102 | 303 | 3.64 |
| 668 | 1073 | 298 | 3.60 |
| 670 | 1044 | 294 | 3.55 |
| 672 | 1016 | 290 | 3.50 |
| 674 | 988 | 286 | 3.46 |
| 676 | 964 | 282 | 3.42 |
| 678 | 940 | 279 | 3.37 |
| 680 | 917 | 278 | 3.30 |

Table 4 herein provides exemplary data for observed values of average photocoagulation threshold energy, in millijoules, for a venous blood mixture, Evein, and an arterial blood mixture, Eartery, as a function of electromagnetic energy wavelength. The standard deviations of these observed values are also provided in Table 4. A presence of full black coagulum in an irradiated sample within a capillary tube, e.g., observed using a dissecting microscope, was used to determine an endpoint (and corresponding threshold energy) for the various samples and irradiation conditions.

Table 4 also provides exemplary values of the calculated threshold energy ratio Eartery/Evein, together with 95% statistical upper and lower bounds for this ratio. For example, a larger observed threshold energy for photocoagulation can indicate that a smaller portion or percentage of the provided energy is being absorbed. Accordingly, the threshold energy can be inversely proportional to the absorptivity of a blood sample. Thus, the ratio Eartery/Evein can be comparable to the absorption selectivity ratio Uvein/Uartery.

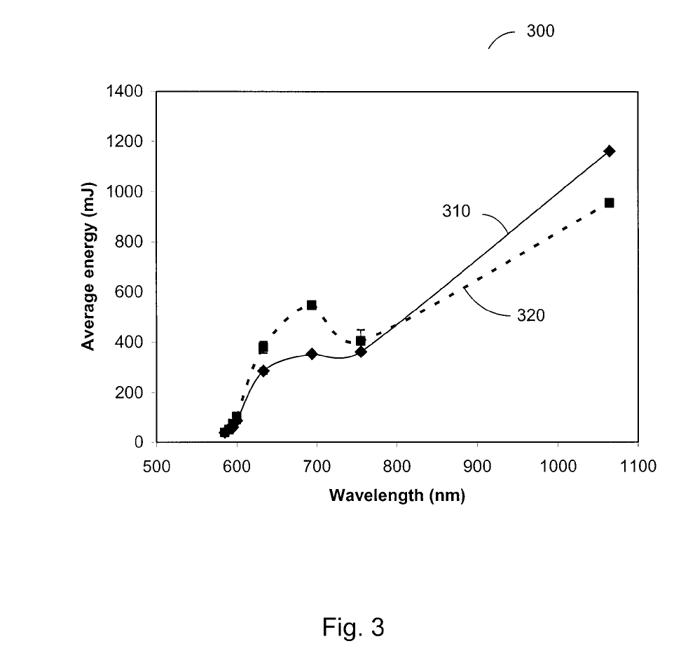

FIG. 3 shows an exemplary graph 300 of the observed values of average photocoagulation threshold energy for a venous blood mixture 310 and an arterial blood mixture 320, which are also provided in Table 4. The exemplary data indicates that the threshold energy 320 of an arterial blood mixture becomes significantly greater than the threshold energy 310 of a venous blood mixture in a range of wavelengths around and just below about 700 nm. These relative energy values can indicate that the venous blood mixture has a higher absorptivity at these wavelengths (e.g., it can absorb a greater proportion of the applied energy, thereby using a lower threshold energy to generate photocoagulation). Accordingly, the absorption selectivity of venous blood as compared to arterial blood can be correspondingly greater at these wavelengths, as indicated by the larger values of the ratio Eartery/Evein.

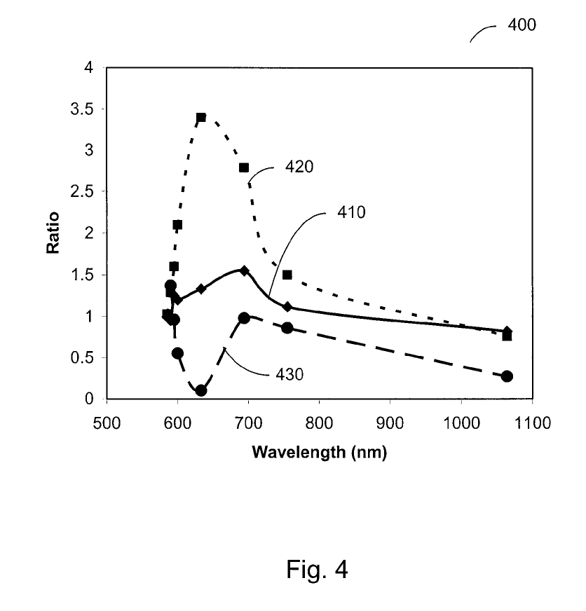

Relative fluence thresholds can be compared with fluence thresholds that may be predicted or estimated based on ratios calculated using HbO2, Hb, and metHb absorption spectra. For example, FIG. 4 shows an exemplary graph 400 illustrating values of the experimentally observed ratio 410 of artery/vein threshold energy over a range of wavelengths of electromagnetic radiation from Table 4. This exemplary ratio 410 can be used as a metric of venous selectivity. For example, a ratio of artery/vein threshold energy that is greater than 1 can indicate a preferential absorption by a vein (e.g., a venous selective condition), and a value of this ratio that is less than 1 can indicate a preferential absorption by an artery (e.g., an artery selective condition).

TABLE 4
Experimental threshold coagulation energies EHb and
EHbO2, and corresponding calculated energy ratio Evein/Eartery.

| Wavelength | Evein (mj) | Eartery (mj) | Eartery/Evein |
|---|---|---|---|
| (nm) | [std. err.] | [std. err.] | [95% bounds] |
| 585 | 39.0 | 38.8 | 0.99 |
| | [1.0] | [0.8] | [0.92-1.07] |
| 590 | 53.2 | 50.7 | 0.95 |
| | [2.2] | [2.0] | [0.84-1.08] |
| 595 | 59.6 | 74.6 | 1.25 |
| | [1.4] | [3.8] | [1.10-1.41] |
| 600 | 86.8 | 104.0 | 1.20 |
| | [2.0] | [2.6] | [1.11-1.29] |
| 633 | 285.8 | 380.3 | 1.33 |
| | [11.9] | [8.7] | [1.21-1.46] |
| 694 | 353.2 | 546.6 | 1.55 |
| | [12.6] | [23.5] | [1.36-1.76] |
| 755 | 362.0 | 404.3 | 1.12 |
| | [5.4] | [10.1] | [1.04-1.20] |
| 1064 | 710.3 | 623.5 | 0.88 |
| | [41.6] | [41.4] | [0.79-0.98] |

FIG. 4 also shows values of a theoretical selectivity ratio 420, Uvein/Uartery, as a function of radiation wavelength. The exemplary ratios 420 can be calculated based on Eq. (5) and on values of UHb and UHbO2 obtained from S. Prahl, Oregeon Medical Laser Center, http://omlc.ogi.edu/spectra/hemoglobin/index.html. A value greater than 1 for this ratio 420 can indicate a venous selectivity, in which the radiation can be preferentially absorbed by a vein as compared to by an artery at a particular wavelength. The ratio values 420 shown in FIG. 4 represent a portion of the data set 210 for such absorption ratios, which can also be provided over a broader range of wavelengths as shown in FIG. 2. As described herein, this selectivity ratio 420 can be determined based, e.g., only on observed absorptivities for Hb and HbO2 (e.g., UHb and UHbO2, respectively), and may not account for any effects of formation of and/or absorption by metHb that may be present.

The exemplary values of the selectivity ratio Uvein/UmetHb 430 shown in FIG. 4 can be calculated using data obtained from W. G. Zijistra et. al., Clin Chem, vol. 37, pp. 1633-1638 (1991), for wavelength-dependent absorption of electromagnetic radiation by metHb. A value greater than 1 for such Uvein/UmetHb ratio 430 can indicate that radiation would be preferentially absorbed by an exemplary venous mixture of 70% HbO2/30% Hb as compared to blood containing only a metHb form of hemoglobin.

The experimental selectivity ratio 410 shown in FIG. 4 (which includes observed absorption behavior where metHb can be present) is generally smaller than the theoretical ratio Uvein/Uartery 420 that can be estimated based on Eq. (5) (which likely does not account for any presence of metHb in blood vessels). The larger values of the observed selectivity ratio 410 also appears to be shifted to somewhat higher wavelengths than the larger values of the theoretical selectivity ratio 420, which does not directly incorporate any effects of metHb that can be present in an irradiated blood sample or vessel. The differences between the exemplary values of the selectivity ratios 410, 420 shown in FIG. 4 can relate to, e.g., certain effects of met-hemoglobin that can be produced during the irradiation of blood samples.

For example, met-hemoglobin that can be produced by irradiation of blood in a blood vessel can affect a net absorptivity of radiation energy by the blood. Met-hemoglobin may exhibit a relatively high absorptivity at wavelengths between about 600 and 660 nm, as indicated the data shown in Table 3. Although the theoretical selectivity ratio Uvein/Uartery 420 exhibits a large value in this exemplary wavelength range, a measured absorptivity ratio 410 can be lower. This lower selectivity can be based in part on a formation of metHb and absorption of radiation by the metHb. For example, metHb can be formed in both veins and arteries by irradiation of blood contained in such blood vessels. The metHb thus formed can absorb further radiation energy. The relatively high absorptivity of metHb can reduce an estimated absorption selectivity between veins and arteries, because metHb, when formed, can absorb a significant amount of radiation in both types of blood vessels.

Such preferential absorption of radiation by metHb as compared to a venous blood mixture is indicated by the curve 430 shown in FIG. 4, which exhibits values significantly less than, e.g., 1 for radiation having a wavelength between about 600 nm and about 700 nm. Accordingly, the formation of metHb having a high absorptivity in an irradiated blood vessel can override a portion of any selectivity effects between arteries and veins that can be based on different absorptivities of Hb and HbO2.

The absorptivity of metHb likely decreases significantly at wavelengths between, e.g., about 680 nm and 700 nm, as provided in Table 3. Accordingly, the presence of any metHb that may be formed upon irradiation of a vessel can have a lesser effect on the overall absorption by the blood contained therein in this wavelength range. This reduced absorptivity of metHb over these wavelengths can thus produce a lesser effect on the estimated vejin/artery selectivity (e.g., a smaller reduction thereof) that is based on the HbHbO2 absorption ratios at these wavelengths.

The exemplary experimental data and exemplary calculations and factors described herein indicate that a highest selectivity of energy absorption for veins as compared with arteries can occur at wavelengths between about 685 nm and about 705 nm, or between about 690 nm and about 700 nm, or at a wavelength of about 694 nm. For example, the experimental data point closest to these wavelength ranges on the curve 410 shown in FIG. 4 is at 694 nm, which was obtained using a ruby laser. This point appears to be close to a local maximum in absorption selectivity as described herein.

Accordingly, exemplary embodiments of the present disclosure can provide methods and apparati for selective treatment of veins, e.g., a photothermolysis treatment, while sparing arteries. Such selective treatment can be achieved, for example, by providing particular electromagnetic radiation for irradiation of veins that can have a wavelength in these selective ranges, e.g., between about 685 nm and about 705 nm, or between about 690 nm and about 700 nm, or about 694 nm.

In one exemplary embodiment of the present disclosure, a method can be provided for treating a vein that can include directing an electromagnetic radiation to a target region of biological tissue, such as skin, containing the vein. Characteristics of the radiation can be chosen so that the radiation can be selectively absorbed by veins as compared with arteries. For example, the exemplary radiation can have a wavelength between about 685 nm and about 705 nm, between about 690 nm and about 700 nm, or about 694 nm. The exemplary radiation can be provided by a pulsed dye laser, a ruby laser, another type of laser, or an intense pulsed light source.

For example, conventional photothermolysis treatments for port wine stains or other vascular lesions can use a pulsed dye laser having a wavelength of about 595 nm.

Use of a radiation source having a longer wavelength of about 685-705 nm, or between about 690 nm and 695 nm, or about 694 nm (e.g., a ruby laser), as described herein, can provide increased selectivity of absorption by veins as compared with arteries. Such longer wavelengths may also facilitate a deeper penetration of the radiation into the target tissue, which can improve treatment efficacy.

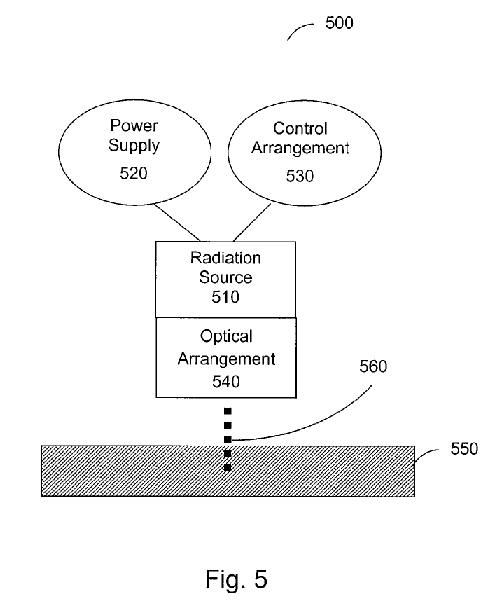

In a further exemplary embodiment of the present disclosure, an exemplary apparatus 500 can be provided, as shown in FIG. 5, for vein-selective photothermolysis treatment. The exemplary apparatus 500 can include a source of electromagnetic radiation 510, a control arrangement 530, and/or an optical arrangement 540. The exemplary apparatus 500 can also include a power source 520. One or more of the components 510-540 can be provided in a single enclosure or a handpiece. Alternatively, one or more of these components 510-540 can be provided in a housing separate from certain other components.

The power source 520 can be structured or configured to provide power to the radiation source 510. The control arrangement 530 can be in wired or wireless, direct or indirect communication with the power source 520 and/or the radiation source 510, and can be configured to control or affect certain properties of the electromagnetic radiation generated by the radiation source 510. The radiation source 510 and the optical arrangement 540 can be structured or configured to direct a particular electromagnetic radiation 560 towards a target region of a biological tissue 550 to be treated and/or effected. For example, the optical arrangement 540 can include, e.g., one or more mirrors or other reflective surfaces, one or more waveguides, e.g., optical fibers or the like, etc.

A cooling arrangement can also be provided to induce a superficial cooling of a portion of the target region of the tissue 550 to be treated and/or affected, e.g., a surface of a target region of the tissue 550. Such cooling can be performed using various conventional techniques and/or arrangements such as, e.g., applying a cryospray or contacting a cooled object to a portion of the target tissue 550.

The target region of the tissue 550 can contain arteries, veins, and/or other structures such as, e.g., other types of blood vessels. The radiation source 510 can be configured to provide the particular electromagnetic radiation 560 having one or more wavelengths that can be preferentially absorbed by the veins as compared to the arteries. For example, the particular radiation 560 can have a wavelength between about 685 nm and about 705 nm, or between about 690 nm and about 700 nm. The radiation source 510 can be a ruby laser that provides electromagnetic radiation at about 694 nm.

The radiation source 510 can include, for example, a pulsed dye laser configured to provide the radiation 560 having a wavelength or plurality of wavelengths that can be preferentially absorbed by veins as compared to arteries, as described herein. Other types of laser that can be configured to emit the radiation 560 at one or more such wavelengths may also be used. Alternatively, an intense pulsed light (IPL) source can be used. The IPL source can be filtered to provide radiation having wavelengths between, e.g., about 685 nm and about 705 nm, as described herein.

Other parameters associated with the particular radiation 560 provided by the radiation source 510 can include, e.g., pulse duration (if a pulsed source is used), fluence, and spot size. These exemplary parameters can be controlled and/or adjusted using the control circuitry 530 when using the exemplary apparatus 500. For example, such parameters can be adjusted to values similar to those that can be used in conventional photothermolysis techniques.

In further exemplary embodiments of the present disclosure, the radiation 560 can be pulsed, with a pulse duration of between about 0.1 ms and about 300 ms, or between about 1 ms and about 300 ms, or between about 10 ms and about 300 ms, or between about 20 ms and about 200 ms. Fluence of the applied electromagnetic radiation 560 (in units of J/cm² or the equivalent thereof) can be selected based on, e.g., the depth and size of a target vein or structure within the targeted tissue. For example, total fluence values of directed or applied radiation 560 may be between about 20 J/cm² and about 80 J/cm².

The parameter values provided herein are exemplary, and other values outside of these ranges can be used depending on the characteristics of the tissue being treated and the desired degree of thermal damage desired. For example, the exemplary parameters of electromagnetic radiation that can be used for photothermolysis of blood vessels in accordance with embodiments of the present disclosure are described, e.g., in U.S. Pat. No. 6,306,130.

Purpura can refer to visible discolorations in skin tissue, e.g., dark spots that may be purplish in color. Purpura can indicate, for example, a presence of bleeding beneath the skin surface where blood may be present locally outside of blood vessels. Such bleeding can be caused, e.g., by exposing blood vessels to electromagnetic radiation to generate thermal damage.

In one exemplary embodiment of the present disclosure, the optical arrangement 540 can include a detector configured to detect purpura in the target region of the tissue 550 when the particular radiation 560 is directed thereon. For example, characteristics of radiation reflected and/or scattered from the tissue 550 can be detected by the optical arrangement 540 using, e.g., conventional signal analysis and/or detection techniques. The control arrangement 530 and/or power source 520 can be configured to shut off or reduce an intensity of the particular radiation 560 provided by the radiation source 510 if purpura is detected in the target region of the tissue 550. This exemplary procedure can reduce a likelihood of generating a non-selective damage to the target tissue 550 that can result from the absorption of the radiation 560 by blood that may be located outside of blood vessels in the target region of the tissue 550.

In a further exemplary embodiment of the present disclosure, the exemplary apparatus 500 may be configured to provide the particular radiation 560 to the target tissue 550 using a plurality of pulses, where each pulse can be of a sufficiently short duration and/or low fluence to avoid formation of purpura within the target tissue 550. Providing a plurality of such “sub-critical” pulses of the radiation 560 can further increase selectivity of absorption of the radiation 560, e.g., by blood vessels in the target tissue 550. A general use of such sub-purpuric pulses of radiation to treat certain blood vessels is described, e.g., in U.S. Pat. No. 5,302,259.

In a still further exemplary embodiment of the present disclosure, the exemplary apparatus 500 can be configured to provide a plurality of pulses of the particular radiation 560 to the target tissue 550, where each successive pulse can have a shorter duration and/or lower fluence than an immediately prior pulse. For example, a plurality of pulses can be provided where a duration of each pulse is less than a duration of the previous pulse. Longer initial pulse durations can be used, e.g., to treat the larger vessels in the target tissue 550, and shorter subsequent pulse durations may treat the smaller vessels. For example, four successive pulses of the radiation 560 can be provided that have exemplary durations of about 10 msec, about 6 msec, about 3 msec, and about 1 msec, respectively. Other numbers of pulses (e.g., 3, 5, or more pulses) can also be used, and the pulse durations used can vary from the exemplary durations described herein.

The foregoing merely illustrates the principles of the present disclosure. Various modifications and alterations to the described embodiments will be apparent to those skilled in the art in view of the teachings herein. It will thus be appreciated that those skilled in the art will be able to devise numerous techniques which, although not explicitly described herein, embody the principles of the present disclosure and are thus within the spirit and scope of the present disclosure. All patents and publications cited herein are incorporated herein by reference in their entireties.

1-32. (canceled)

33. A method for treating at least one venous structure, comprising:
providing a particular electromagnetic radiation to a tissue region containing the at least one venous structure,
wherein the particular radiation has at least one property which effects the particular radiation to be more effectively absorbed by the at least one venous structure than by an artery present in the tissue region, and
wherein the particular radiation has a wavelength between about 685 nm and about 705 nm.

34. The method of claim 33, wherein the particular radiation has a wavelength between about 690 nm and about 700 nm.

35. The method of claim 33, wherein the particular radiation has a wavelength of about 694 nm.

36. The method of claim 33, wherein the particular radiation is provided using at least one of a pulsed dye laser, a wavelength-shifted Nd:YAG laser, a frequency-doubled infrared laser, a high power diode laser array, or a fiber laser.

37. The method of claim 33, wherein the particular radiation is provided using a ruby laser.

38. The method of claim 33, wherein the particular radiation is provided using an intense pulsed light source.

39. The method of claim 38, further comprising filtering the radiation that is provided by the intense pulsed light source.

40. The method of claim 33, wherein the at least one venous structure is at least one of a port wine stain or a varicose vein.

41. The method of claim 33, wherein the particular electromagnetic radiation comprises a plurality of pulses, and wherein at least one of a duration or a fluence of each of the pulses is selected to avoid a formation of purpura in the tissue region.

42. The method of claim 33, wherein the particular electromagnetic radiation comprises a plurality of pulses, and wherein at least one of a duration or a fluence of each of the pulses is less than a duration or fluence of a preceding one of the pulses.

43. An apparatus for treating at least one venous structure, comprising:
a radiation source configured to provide a particular electromagnetic radiation to a biological tissue containing the at least one venous structure,
wherein the particular radiation has at least one property which effects the particular radiation to be more effectively absorbed by the at least one venous structure than by an artery present in the tissue region, and
wherein the particular radiation has a wavelength between about 685 nm and about 705 nm.

44. The apparatus of claim 43, wherein the particular radiation has a wavelength between about 690 nm and about 700 nm.

45. The apparatus of claim 43, wherein the particular radiation has a wavelength of about 694 nm.

46. The apparatus of claim 43, further comprising an optical arrangement structured to provide the particular radiation to at least one portion of the biological tissue.

47. The apparatus of claim 43, further comprising a control arrangement structured to at least one of control or adjust the at least one property of the particular radiation.

48. The apparatus of claim 43, wherein the at least one property is at least one of a pulse duration, a fluence, an intensity, or a wavelength.

49. The apparatus of claim 43, wherein the radiation source comprises at least one of a pulsed dye laser, a wavelength-shifted Nd:YAG laser, a frequency-doubled infrared laser, a high power diode laser array, a fiber laser, or an intense pulsed light source.

50. The apparatus of claim 43, wherein the radiation source comprises an intense pulsed light source and a filter in communication with the intense pulsed light source.

51. The apparatus of claim 43, wherein the radiation source is configured to provide the particular electromagnetic radiation as a plurality of pulses, and wherein at least one of a duration or a fluence of each of the pulses is selectable to avoid a formation of purpura in the tissue region.

52. The apparatus of claim 43, wherein the radiation source is configured to provide the particular electromagnetic radiation as a plurality of pulses, and wherein at least one of a duration or a fluence of each of the pulses is less than the duration or the fluence of a preceding one of the pulses.
